(19) United States
(12) Patent Application Publication
(10) Pub. No.: US 2019/0307993 A1
(43) Pub. Date: Oct. 10, 2019

(54) Low Profile Balloon Catheter And Related Systems And Methods

(71) Applicant: QXMedical, LLC, Roseville, MN (US)

(72) Inventors: Fernando Di Caprio, St. Paul, MN (US); Gianfranco Panarello, Mount Royal (CA)

(21) Appl. No.: 16/375,280

(22) Filed: Apr. 4, 2019

Related U.S. Application Data
(60) Provisional application No. 62/652,592, filed on Apr. 4, 2018.

(51) Int. Cl. A61M 25/10

(52) U.S. Cl. CPC A61M 25/10184; A61M 25/1002; A61M 25/09; A61M 2025/1056; A61M 25/1025

(57) ABSTRACT

Disclosed herein are various balloon catheter embodiments having a reduced profile (or reduced diameter) section along a length of the catheter body and an inflatable body disposed over the reduced profile section. In certain embodiments, the catheter body has at least one inflation/deflation lumen and a distal inflation/deflation opening defined in a proximal lip of the reduced profile section.

### CROSS-REFERENCE TO RELATED APPLICATION(S)

This application claims the benefit under 35 U.S.C. § 119(e) to U.S. Provisional Application 62/652,592, filed Apr. 4, 2018 and entitled “Low Profile Balloon Catheter and Related Systems and Methods,” which is hereby incorporated herein by reference in its entirety.

### FIELD OF THE INVENTION

The various embodiments disclosed or contemplated herein relate to intervention technologies, including cardiopulmonary intervention technologies, and more specifically to balloon catheters and related systems and methods.

### BACKGROUND OF THE INVENTION

Known balloon catheters, including, for example, the known catheter 10 depicted in FIGS. 1A and 1B, typically have a catheter body 12 with an inflatable body 14 disposed thereon. Generally, the catheter body 12 has three lumens, including a guidewire lumen 16 and two inflation/deflation lumens 18, 20. Additional lumens may be incorporated for a number of other purposes, such as, for example, additional inflation/deflation capacity, injection of procedural fluids, sampling of biological fluids, passage of devices, measurement of pressure, etc. It is understood that the inflatable body 14 disposed on the outer surface of the catheter body 12 typically results in the catheter 10 having a larger diameter or radial cross-section along the length of the catheter body 12 around which the inflatable body 14 is disposed, even when the inflatable body 14 is in its deflated configuration.

During a typical procedure, the balloon catheter 10 is inserted through a known introducer sheath (not shown) that provides transvenous or arterial access to advance the catheter 10 to a target location within a blood vessel. Once the balloon catheter 10 is positioned as desired, the inflatable body 14 can be inflated by providing an inflation fluid (such as ambient air or any other inflation fluid) through the inflation/deflation lumens 18, 20 such that the inflation fluid enters the inflatable body via the inflation/deflation openings 22, 24, each of which is in fluidic communication with one of the inflation/deflation lumens 18, 20. After the procedure is complete, the inflatable body 14 is deflated by allowing the inflation fluid to pass proximally out through the inflation/deflation lumens 18, 20 via the inflation/deflation openings 22, 24, until the inflatable body 14 has returned to its deflated configuration.

One disadvantage of known balloon catheters such as the catheter 10 of FIGS. 1A and 1B is that the need for lumens (such as lumens 16, 18, 20) defined through the catheter body (such as body 12) and the need for an inflatable body (such as inflatable body 14) attached or otherwise disposed on an outer surface of the catheter body (such as body 12) results in a relatively larger diameter or radial cross-section (in comparison to catheters that don't require inflatable bodies) along at least some length of the catheter body, especially along the length of the catheter body where the inflatable body is disposed, which makes a larger introducer sheath necessary in order to receive the catheter.

There is a need in the art for an improved balloon catheter with a relatively smaller radial cross-section (or lower profile) along at least a length of the catheter body.

### BRIEF SUMMARY OF THE INVENTION

Discussed herein are various balloon catheter embodiments having a reduced profile or reduced diameter length.

In Example 1, a balloon catheter comprises a catheter body comprising at least one non-reduced profile body section, and a reduced profile body section disposed proximal of a distal end of the catheter body. Further, the balloon catheter also comprises an inflatable body disposed over the reduced profile body section, a guidewire lumen disposed through the standard body section and the reduced profile body section, at least one inflation/deflation lumen disposed through a proximal portion of the standard body section, and at least one distal inflation/deflation opening in fluid communication with the at least one inflation/deflation lumen, wherein the at least one inflation/deflation opening is disposed within the inflatable body.

Example 2 relates to the balloon catheter according to Example 1, wherein the reduced profile body section has a diameter ranging from about 10% to about 75% of the at least one non-reduced profile body section.

Example 3 relates to the balloon catheter according to Example 1, wherein the reduced profile body section has a diameter ranging from about 30% to about 60% of the at least one non-reduced profile body section.

Example 4 relates to the balloon catheter according to Example 1, wherein at least one of the at least one non-reduced profile body sections is disposed proximal of the reduced profile body section.

Example 5 relates to the balloon catheter according to Example 1, wherein a first non-reduced profile body section is disposed proximal of the reduced profile body section and a second non-reduced profile body section is disposed distal of the reduced profile body section.

Example 6 relates to the balloon catheter according to Example 1, further comprising a proximal lip at a proximal end of the reduced profile body section.

Example 7 relates to the balloon catheter according to Example 6, wherein the at least one distal inflation/deflation opening is defined in the proximal lip.

In Example 8, a balloon catheter comprises a proximal catheter body section, a reduced profile body section distal of the proximal catheter body section, a distal catheter body section distal of the reduced profile body section, an inflatable body disposed over the reduced profile body section, a guidewire lumen disposed through the proximal catheter body section, the reduced profile body section and the distal catheter body section, and at least one inflation/deflation lumen disposed through the proximal catheter body section and in fluid communication with an interior of the inflatable body.

Example 9 relates to the balloon catheter according to Example 8, wherein the reduced profile body section has a diameter ranging from about 10% to about 75% of one of the proximal catheter body section and the distal catheter body section.

Example 10 relates to the balloon catheter according to Example 8, wherein the reduced profile body section has a diameter ranging from about 30% to about 60% of one of the proximal catheter body section and the distal catheter body section.

Example 11 relates to the balloon catheter according to Example 8, further comprising a proximal lip at a proximal end of the reduced profile body section.

Example 12 relates to the balloon catheter according to Example 11, wherein the at least one inflation/deflation lumen comprises a distal inflation/deflation opening in fluid communication with the at least one inflation/deflation lumen, wherein the distal inflation/deflation opening is defined in the proximal lip.

In Example 13, a balloon catheter comprises a catheter body comprising a first body section having a first diameter, a second body section disposed proximal to the first body section and having a second diameter, wherein the second diameter is smaller than the first diameter, and a third body section disposed proximal to the second body section and having a third diameter, wherein the third diameter is substantially equal to the first diameter. Further, the balloon catheter also comprises an inflatable body disposed over the second body section, a guidewire lumen defined through the first, second, and third body sections, first and second inflation/deflation lumens defined through the first body section, a first opening in fluid communication with the first inflation/deflation lumen, wherein the first opening is disposed within the inflatable body such that the first inflation/deflation lumen is in fluid communication with an interior of the inflatable body, and a second opening in fluid communication with the second inflation/deflation lumen, wherein the second opening is disposed within the inflatable body such that the second inflation/deflation lumen is in fluid communication with the interior of the inflatable body.

Example 14 relates to the balloon catheter according to Example 13, wherein the second diameter is a diameter ranging from about 10% to about 75% of the first diameter.

Example 15 relates to the balloon catheter according to Example 13, wherein the second diameter is a diameter ranging from about 30% to about 60% of the first diameter.

Example 16 relates to the balloon catheter according to Example 13, wherein the second diameter is a diameter of about 50% of the first diameter.

Example 17 relates to the balloon catheter according to Example 13, further comprising a proximal lip at a proximal end of the second body section.

Example 18 relates to the balloon catheter according to Example 17, wherein the first and second openings are defined in the proximal lip.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a side view of a standard balloon catheter.

FIG. 1B is a cross-sectional, cutaway view of the catheter body of the balloon catheter of FIG. 1A.

FIG. 2A is a side view of a balloon catheter with a reduced diameter section, according to one embodiment.

FIG. 2B is a cross-sectional cutaway view of the body of the balloon catheter of FIG. 2A, according to one embodiment.

FIG. 3A is a cross-sectional cutaway view of the body of a balloon catheter with a reduced diameter section, according to another embodiment.

FIG. 3B is a cross-sectional cutaway view of the body of a balloon catheter with a reduced diameter section, according to another embodiment.

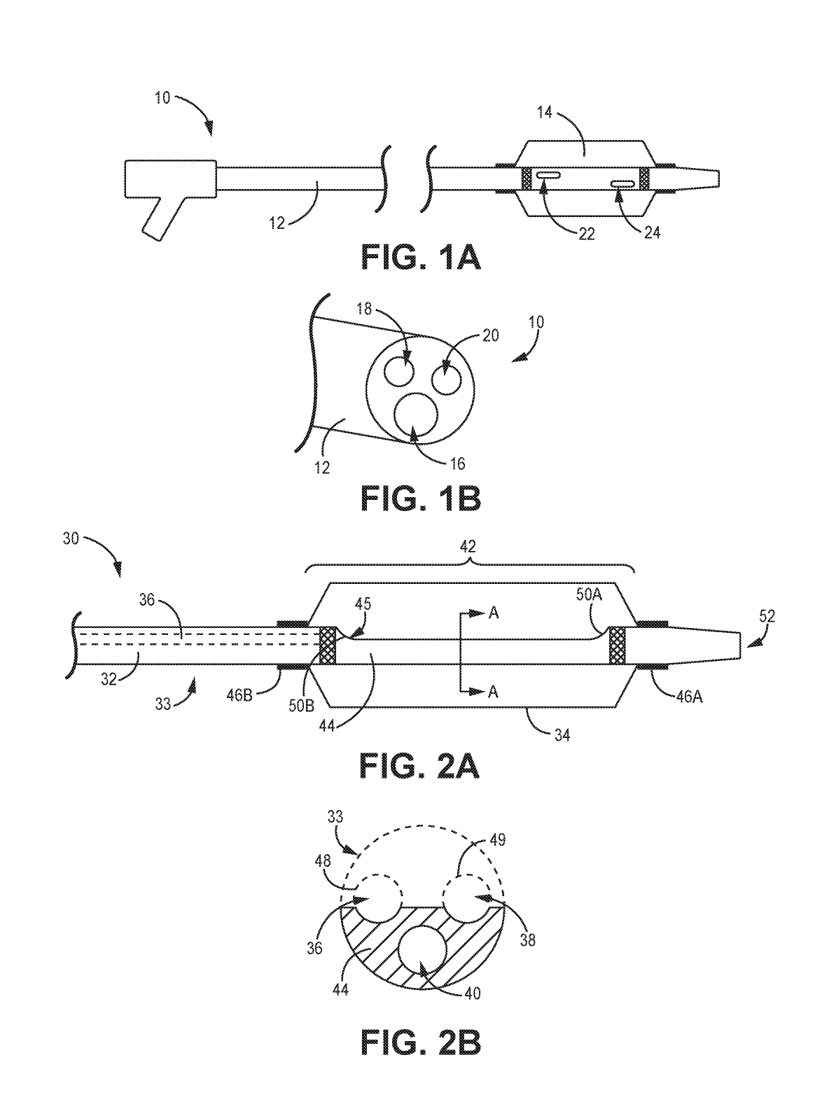

### DETAILED DESCRIPTION

The various embodiments disclosed or contemplated herein relate to improved balloon catheters, in which each such catheter has a reduced radial cross-section (also referred to herein as a “lower profile” or “smaller diameter”) along a length of the catheter body. In certain implementations, the reduced diameter length or section is disposed along the length of the catheter body around or along which the inflatable body is disposed (also referred to herein as the “balloon section,” “inflatable body section,” “balloon zone,” or “inflatable body zone”).

One balloon catheter 30 with a reduced diameter section according to one embodiment is depicted in FIGS. 2A and 2B. The catheter 30 has a catheter body 32, an inflatable body 34, inflation/deflation lumens 36, 38, and a guidewire lumen 40. In addition, in the inflatable body zone 42, the catheter body 32 has a reduced diameter section or segment (also referred to as a reduced or low profile section or segment) 44 in which the body 32 has a smaller diameter or smaller radial cross-section in comparison to the rest of the length of the catheter body 32. The reduced diameter section 44, according to one embodiment, is the entire, or a substantial amount of the, section or length of the catheter body 32 within the inflatable body zone 42. Alternatively, the reduced diameter section 44 is the section or length between the distal inflation body attachment point 46A and the proximal inflation body attachment point 46B. In a further alternative, the reduced diameter section 44 can be any length of the catheter body that is within the inflatable body zone 42, including about 50% of the length, less than 50% of the length, or greater than 50% of the length of the catheter body within the inflatable body zone 42.

According to one implementation as shown in FIGS. 2A and 2B, the reduced diameter section 44 has an edge (or “lip”) 50A, 50B at each end of the section 44. That is, the distal edge 50A forms the distal end of the reduced diameter section 44 while the proximal edge 50B forms the proximal end of the reduced diameter section 44. The distal and proximal edges 50A, 50B can be lips, walls, or any other known features or formations that separate the larger catheter body 32 from the reduced diameter section 44 and extend therebetween.

In this embodiment, the inflation/deflation lumens 36, 38 both extend from a proximal end (not shown) of the catheter 30 along a length of the catheter body 32 and have distal openings defined in the body 32 that are disposed within the inflatable body 34. However, instead of the lumens 36, 38 extending some length along the catheter body 32 within the inflatable body 34 to openings positioned in a fashion similar to the openings 22, 24 discussed above with respect to the standard catheter 10, the two lumens 36, 38 in this exemplary implementation extend only to the proximal edge 50B of the reduced diameter section 44. That is, the distal inflation/deflation openings 48, 49 are defined in the proximal edge 50B as best shown schematically in FIG. 2B.

It is understood that because the inflation/deflation lumens 36, 38 extend from the proximal end (not shown) to the proximal edge 50B of the reduced diameter section 44, there is no need to reduce the inner diameter of those lumens 36, 38. That is, the lumens 36, 38 are defined within the length of the body 32 proximal to the reduced diameter section 44 that is not reduced in diameter, which is referred to herein as the “full diameter section” (also referred to as a “non-reduced diameter section,” a “standard body section,” and a “standard diameter section”) 33. Because the lumens 36, 38 are disposed within and extend along the full diameter section 33 of the body 32, but not the reduced diameter section 44, the lumens 36, 38 can remain at “full” size as well, thereby maintaining the full inflation/deflation capability of each lumen 36, 38.

FIG. 2B is a cross-sectional view of the reduced diameter section 44 that specifically depicts the portion of the reduced diameter section 44 along line A in FIG. 2A. It provides a perspective of the cross-section of the reduced diameter section 44 such that the longitudinal axis of the catheter body 32 is transverse to the plane of the page. FIG. 2B also depicts the dimensions of the full diameter section 33 with dashed lines to provide a comparison of the diameter of the full diameter section 33 to the reduced diameter or profile of the reduced diameter section 44. Further, the figure provides a schematic perspective of the inflation/deflation openings 48, 49.

According to one implementation, in contrast to the inflation/deflation lumens 36, 38, the guidewire lumen 40 is defined in both the full diameter section 33 and the reduced diameter section 44 such that guidewire lumen 40 extends from the proximal end (not shown) to the distal end 52 of the catheter 30, as best shown in FIG. 2B. For most procedures, it is beneficial, if not necessary, for the guidewire lumen 40 to extend from the proximal end (not shown) to the distal end 52.

The full (or non-reduced) diameter section, according to any of the embodiments disclosed or contemplated herein, can have a diameter that is the same as the largest diameter of the body of any standard balloon catheter. For example, in one embodiment, the full diameter section can have a size ranging from about 3 French to about 26 French. Alternatively, the full diameter section can have a size ranging from about 4 French to about 16 French. In a further alternative, the full diameter section can have a size ranging from about 5 French to about 9 French.

In the various implementations disclosed or contemplated herein, the diameter of the reduced diameter section (such as section 44) is less than the diameter of the full diameter section (such as section 33) by a predetermined amount. For example, in one embodiment, the reduced diameter section has a diameter ranging from about 10% to about 75% of the diameter of the full diameter section. Alternatively, the reduced diameter section has a diameter ranging from about 35% to about 65% of the diameter of the full diameter section. In a further alternative, the reduced diameter section has a diameter ranging from about 45% to about 55% of the diameter of the full diameter section. In yet another implementation, the reduced diameter has a diameter of about 50% of the diameter of the full diameter section.

In one embodiment, the reduced diameter section 44 can be formed by simply making the catheter 30 with the reduced diameter section 44 already formed in the catheter 30. Alternatively, the reduced diameter section 44 can subsequent be machined out of or otherwise physically removed from the catheter 30 after initial construction of the catheter 30.

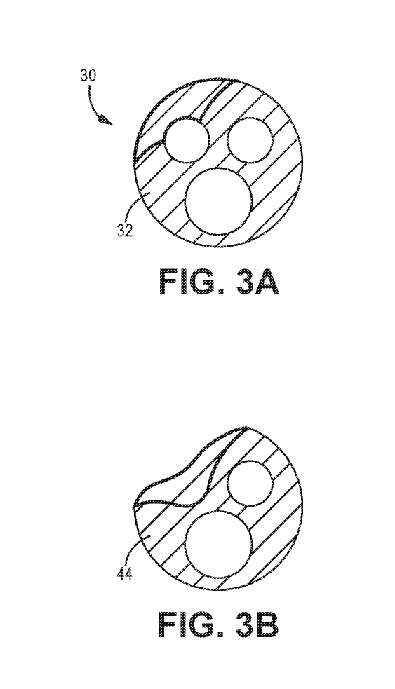

According to another alternative, rather than initially forming the reduced diameter section or removing material to form the reduced diameter section, as best shown in FIGS. 3A and 3B, the reduced diameter section 44 can also be formed by physically collapsing or crushing or otherwise physically reducing the diameter of the catheter 30 to form the reduced diameter section 44. That is, instead of removing material from the catheter 30, a desired portion of the catheter body 32 is melted, pressed, ironed, crushed, or otherwise physically reduced to form the reduced diameter section 44.

In accordance with any of the embodiments disclosed or contemplated herein, one benefit of the reduced diameter section is that the section provides additional space (in comparison to the full diameter section of the catheter body) for the deflated inflatable body material to be positioned during insertion of the catheter through an introducer sheath or similar introduction device, thereby making it possible for a smaller introducer sheath to be used. That is, the reduced diameter section (such as section 44 above, for example) has a reduced profile in comparison to the full diameter section of the body, thereby providing additional space along the length of the reduced diameter section between the outer surface of the reduced diameter section and the inner surface of the sheath and/or blood vessel. As a result, the deflated inflatable body material can be positioned within that additional space, thereby reducing the need for a larger sheath with more space between the outer surface of a known balloon catheter body with a standard diameter and the inner surface of the sheath.

Another benefit, according to a further embodiment, is that the reduced amount of material in the catheter body 32 along the reduced diameter section 44 results in the reduced diameter section 44 being more flexible than the full diameter section 33. As such, the length or section of the catheter 30 proximal of the expandable body zone 42 has sufficient stiffness/pushability/stability to allow a user to urge the catheter body 32 into and through a sheath and/or a target blood vessel while a distal portion of the catheter 30 extending from the expandable body zone 42 to the distal end 52 has greater flexibility. As such, the increased flexibility of the distal portion of the device 30 improves the trackability or steerability of the catheter 30 by improving the ability to use the device 30 to navigate tortuous vessels.

Although the present invention has been described with reference to preferred embodiments, persons skilled in the art will recognize that changes may be made in form and detail without departing from the spirit and scope of the invention.

What is claimed is:

1. A balloon catheter comprising:
(a) a catheter body comprising:
(i) at least one non-reduced profile body section; and
(ii) a reduced profile body section disposed proximal of a distal end of the catheter body;
(b) an inflatable body disposed over the reduced profile body section;
(c) a guidewire lumen disposed through the standard body section and the reduced profile body section;
(d) at least one inflation/deflation lumen disposed through a proximal portion of the standard body section; and
(e) at least one distal inflation/deflation opening in fluid communication with the at least one inflation/deflation lumen, wherein the at least one inflation/deflation opening is disposed within the inflatable body.

2. The balloon catheter of claim 1, wherein the reduced profile body section has a diameter ranging from about 10% to about 75% of the at least one non-reduced profile body section.

3. The balloon catheter of claim 1, wherein the reduced profile body section has a diameter ranging from about 30% to about 60% of the at least one non-reduced profile body section.

4. The balloon catheter of claim 1, wherein at least one of the at least one non-reduced profile body sections is disposed proximal of the reduced profile body section.

5. The balloon catheter of claim 1, wherein a first non-reduced profile body section is disposed proximal of the reduced profile body section and a second non-reduced profile body section is disposed distal of the reduced profile body section.

6. The balloon catheter of claim 1, further comprising a proximal lip at a proximal end of the reduced profile body section.

7. The balloon catheter of claim 6, wherein the at least one distal inflation/deflation opening is defined in the proximal lip.

8. A balloon catheter comprising:
(a) a proximal catheter body section;
(b) a reduced profile body section distal of the proximal catheter body section;
(c) a distal catheter body section distal of the reduced profile body section;
(d) an inflatable body disposed over the reduced profile body section;
(e) a guidewire lumen disposed through the proximal catheter body section, the reduced profile body section and the distal catheter body section; and
(f) at least one inflation/deflation lumen disposed through the proximal catheter body section and in fluid communication with an interior of the inflatable body.

9. The balloon catheter of claim 8, wherein the reduced profile body section has a diameter ranging from about 10% to about 75% of one of the proximal catheter body section and the distal catheter body section.

10. The balloon catheter of claim 8, wherein the reduced profile body section has a diameter ranging from about 30% to about 60% of one of the proximal catheter body section and the distal catheter body section.

11. The balloon catheter of claim 8, further comprising a proximal lip at a proximal end of the reduced profile body section.

12. The balloon catheter of claim 11, wherein the at least one inflation/deflation lumen comprises a distal inflation/deflation opening in fluid communication with the at least one inflation/deflation lumen, wherein the distal inflation/deflation opening is defined in the proximal lip.

13. A balloon catheter comprising:
(a) a catheter body comprising:
(i) a first body section having a first diameter;
(ii) a second body section disposed proximal to the first body section and having a second diameter, wherein the second diameter is smaller than the first diameter;
(iii) a third body section disposed proximal to the second body section and having a third diameter, wherein the third diameter is substantially equal to the first diameter;
(b) an inflatable body disposed over the second body section;
(c) a guidewire lumen defined through the first, second, and third body sections;
(d) first and second inflation/deflation lumens defined through the first body section;
(e) a first opening in fluid communication with the first inflation/deflation lumen, wherein the first opening is disposed within the inflatable body such that the first inflation/deflation lumen is in fluid communication with an interior of the inflatable body; and
(f) a second opening in fluid communication with the second inflation/deflation lumen, wherein the second opening is disposed within the inflatable body such that the second inflation/deflation lumen is in fluid communication with the interior of the inflatable body.

14. The balloon catheter of claim 13, wherein the second diameter is a diameter ranging from about 10% to about 75% of the first diameter.

15. The balloon catheter of claim 13, wherein the second diameter is a diameter ranging from about 30% to about 60% of the first diameter.

16. The balloon catheter of claim 13, wherein the second diameter is a diameter of about 50% of the first diameter.

17. The balloon catheter of claim 13, further comprising a proximal lip at a proximal end of the second body section.

18. The balloon catheter of claim 17, wherein the first and second openings are defined in the proximal lip.
